(19) United States
(12) Patent Application Publication
(10) Pub. No.: US 2012/0077888 A1
(43) Pub. Date: Mar. 29, 2012

(54) ORODISPERSIBLE TABLETS

(75) Inventors: Zeibunissa Ramtoola, Dublin (IE); Ritesh Pabari, Dublin (IE); Asha Jamil, Dublin (IE)

(73) Assignee: ROYAL COLLEGE OF SURGEONS IN IRELAND, Dublin 2 (IE)

(21) Appl. No.: 13/260,384

(22) PCT Filed: Mar. 26, 2010
(86) PCT No.: PCT/EP10/54047

§ 371 Date: Dec. 16, 2011

Related U.S. Application Data
(60) Provisional application No. 61/163,648, filed on Mar. 26, 2009.

(30) Foreign Application Priority Data
Mar. 26, 2009 (EP) 09156370.0

(51) Int. Cl. A61K 47/38; B29C 59/02

(52) U.S. Cl. 514/781; 264/122

(57) ABSTRACT

A directly compressed orodispersible tablet comprises 0.1 to 50% of a ungranulated active agent (w/w), 10 to 80% of a sugar-based direct compression base, and 10 to 80% of a microcrystalline cellulose (MCC) direct compression base, and has a hardness of at least 60N, and a disintegration time of less than 40 seconds. The sugar-based direct compression base is a DC sugar alcohol, especially direct compression mannitol, and the MCC base is a silicified MCC, especially a Prosolv. The active is a hydrophobic active, typically a high-dose active. Also disclosed is a method of producing an orodispersible tablet comprising the steps of directly compressing a mixture of components at a compression force of at least 5 k N to form the tablet, wherein the mixture of components comprises 0.1 to 50% of an active agent (w/w), 10 to 80% of a sugar-based direct compression base (w/w); and 10 to 80% of a microcrystalline cellulose (MCC) direct compression base (w/w).

### TECHNICAL FIELD

The invention relates to directly compressed orodispersible tablets, and method for the production thereof. In particular, the invention relates to directly compressed orodispersible tablets comprising a hydrophobic active.

### BACKGROUND TO THE INVENTION

The use of conventional tablets is often challenging to geriatric, paediatric and uncooperative patients who have difficulties swallowing. Further, swallowing conventional tablets can be a problem when patients have a persistent cough or a gag-reflex, or when water is unavailable. These problems have been partly addressed by the provision of fast dissolving tablets in recent years. These tablet forms are also known as FDDT (fast dissolving disintegration tablets), fast melt, or oral dissolving, tablets. Generally, these tablets include one or more hydrophilic disintegrants that, when placed on the tongue or in the oral cavity, rapidly absorb saliva and dissolve or disperse within less than one minute. A problem with the provision of these tablets is the need to provide a tablet that is sufficiently strong to withstand packaging, transport, and subsequent handling without breaking, yet capable of disintegrating rapidly when placed in the oral cavity. This problem has been addressed in a number of ways.

WO2004/091585 published in the name of Synthon BV, discloses the use of silicified microcrystalline cellulose as a matrix forming excipient in the composition of fast melt tablets. These tablets have a reported hardness of approximately 30N/40N and a disintegration time of 30 seconds. Biovail Technologies (WO2004/000197) have addressed the issue of producing a suitably robust tablet by developing a process that combines direct compression of Liquiflash™ microspheres with an excipient; for example a compressible inorganic salt or a cellulose derivative. These tablets are capable of dissolving in less than 40 seconds and have a hardness of 20N to 37N. The manner in which these tablets are produced eliminates the need for complex processing techniques and equipment. WO2006/002937 (Lek Pharmaceuticals) discloses orodispersible tablets containing non-filamentous co-processed polyol particles that are produced by spray drying, silicified microcrystalline cellulose, and an active agent that needs to be granulated prior to formation of the tablet by direct compression. The purpose of the silicified microcrystalline cellulose in the mix is to address a problem of segregation which occurred due to the large particle size difference between the granulated active and the spray-dried excipient particles. The requirement to provide the active agent in a granulated form is technologically demanding and requires specialised processing prior to blending and tableting.

It is an object of the invention to overcome at least one of the above-mentioned problems.

### STATEMENTS OF INVENTION

In one aspect, the invention relates to a method of producing orodispersible tablets having low disintegration times, for example less than 60, 50, 40, 30, 20, 18, or 17 seconds, high hardness, for example at least 50, 55, or 60N, which employs at least two direct compression excipients including a microcrystalline cellulose and a sugar-based direct compression excipient, and an active which is not required to be in a granulated form. The method of the invention suitably involves dry-blending these components and directly compressing the blend using relatively high compression forces, for example at least 5, 6, 7, 8, 9, 10, 11 or 12 kN, to produce the orodispersible tablets. The Applicant has surprisingly discovered that the use of non-granulated active in combination with at least two direct compression excipients, including a microcrystalline cellulose and a sugar-based direct compression excipient results in highly robust tablets having very low disintegration times. The use of granulated actives can retard the dissolution of the active and hence its bioavailability from the tablet, as the tablet is first required to disintegrate to release the granulated active, and then the granules are required to disintegrate/dissolve before the active is released. In the method of the present invention, the active is provide in a non-granulated form, which is suitably dry blended with two direct compression excipients before tabletting, and this has been found to produce tablets having low disintegration times and high hardness values.

Thus, in one aspect, the invention relates to a method of producing an orodispersible tablet comprising the steps of directly compressing a mixture of components at a compression force of at least 5, 6, 7 or 8 kN to form the tablet, wherein the mixture of components comprises 0.1 to 50% of an active agent (w/w), 10 to 80% of a sugar-based direct compression base (w/w); and 10 to 80% of a microcrystalline cellulose (MCC) direct compression base (w/w).

The invention also relates to a method of producing an orodispersible tablet comprising the steps of directly compressing a mixture of components at a compression force of at least 5, 6, 7 or 8 kN to form the tablet, wherein the mixture of components comprises 0.1 to 30% of an active agent (w/w), 30 to 70%, 30 to 60%, or 30 to 50%, of a sugar-based direct compression base (w/w); and 30 to 70%, 30 to 60%, or 30 to 50%, of a microcrystalline cellulose (MCC) direct compression base (w/w).

Typically, the tablets are directly compressed at a compression force of at least 9 kN, 10 kN, 11 kN or 12 kN. Suitably, the tablets are directly compressed using flat-faced toolings.

Typically, the tablets producible by the process have a hardness of at least 50N, ideally at least 60N, and suitably a disintegration time of less than 60, 50, 40, 30, 20, or 18 seconds. In one embodiment, the MCC direct compression base is a silicified MCC direct compression base, and in which the tablets producible by the process have a disintegration time of less than 20 seconds.

Suitably, the mixture of components comprises 0.1 to 30% of an active agent (w/w), 40 to 50% of a sugar-based direct compression base and 40 to 50% of a microcrystalline cellulose (MCC) direct compression base.

Typically, the sugar-based direct compression excipient is not a co-processed mixture of two sugar alcohols, for example a solution comprising a mixture mannitol and sorbitol which is spray-dried.

In a related aspect, the invention relates to orodispersible tablets that are capable of dissolving rapidly in the oral cavity, for example in a time of less than 60, 50, 40, 30, 25, 20 or 18 seconds, and yet are sufficiently hard to be packaged in conventional packaging, for example having a hardness of at least 50N or 60N. Briefly, the tablets are formed by direct compression (i.e. directly compressed tablets), and include an active agent, often a hydrophobic active agent, which generally has an average particle size of less than 100μ. The tablets also include a sugar-based direct compression base, for example a direct compression sugar-based excipient such as a sugar or a sugar alcohol such as mannitol which provides palatability, processability, and typically comprises particles having an average size of greater that 100μ. Surprisingly, it has been discovered that the flow characteristics of the tablet components, and the hardness and/or disintegration times, are improved by inclusion of a second direct compression base having particles that are closer in size to the particles of active. A microcrystalline cellulose (MCC) base, especially a silicified MCC such as ProSolv (WO96/21429) which typically comprises particles having an average size of less than 100μ, has been found to provide excellent properties when combined with a sugar-based DC base, especially when formulated with a high dose active. In addition, the Applicant has surprisingly discovered that the bioavailability of the active is improved by providing the active in a non-granulated form.

Thus, according to a further aspect of the invention, there is provided a directly compressed orodispersible tablet comprising:
0.1 to 50% of a non-granulated active agent (w/w);
10 to 80% of a sugar-based direct compression base (w/w); and
10 to 80% of a microcrystalline cellulose (MCC) direct compression base (w/w).

In this specification, the term “orodispersible tablet” should be taken to mean that the tablet has a disintegration time of 60 seconds or less. Typically, the tablets have a hardness of at least 50N or 60N, and a disintegration time of less than 60 seconds. Preferably, the tablets have a disintegration time of less than 40 seconds and a hardness of at least 60N. Ideally, the tablets have a disintegration time of less than 20 seconds and a hardness of at least 60N.

The invention relates to a directly compressed orodispersible tablet according to the invention and comprising:
0.1 to 20% of a non-granulated active agent (w/w);
30 to 70%, 30 to 60%, or 30 to 50%, of a DC sugar alcohol (w/w);
30 to 70%, 30 to 60%, or 30 to 50%, of a silicified MCC (w/w); and
optionally, one or more of a lubricant, a disintegrant, flavouring agent, and a flow enhancer,
wherein the tablet has a hardness of at least 50N, preferably at least 60N, and a disintegration time of less than 60 seconds, preferably less than 40, 30, 20 or 18 seconds.

The invention relates to a directly compressed orodispersible tablet according to the invention and comprising:
0.1 to 20% of a (typically hydrophobic) non-granulated active agent (w/w);
30 to 50% of a DC sugar alcohol (w/w);
30 to 50% of a silicified MCC (w/w);
1 to 20% of a disintegrant, ideally a superdisintegrant (w/w); and
optionally, one or more of a flavouring agent, and a flow enhancer,
wherein the tablet has a hardness of at least 50N, preferably at least 60N, and a disintegration time of less than 60 seconds, preferably less than 40, 30, 20 or 18 seconds.

The invention relates to a directly compressed orodispersible tablet according to the invention and consisting essentially of:
0.1 to 50% of a non-granulated active agent (w/w);
10 to 80% of a DC sugar alcohol (w/w);
10 to 80% of a silicified MCC (w/w); and
optionally, one or more of a lubricant, a disintegrant, flavouring agent, and a flow enhancer,
wherein the tablet has a hardness of at least 50N, preferably at least 60N, and a disintegration time of less than 60 seconds, preferably less than 40, 30, 20 or 18 seconds.

The invention relates to a directly compressed orodispersible tablet according to the invention and consisting essentially of:
0.1 to 30% of a non-granulated active agent (w/w);
30 to 50% of a DC sugar alcohol (w/w);
30 to 50% of a silicified MCC (w/w); and
optionally, one or more of a lubricant, a disintegrant, flavouring agent, and a flow enhancer,
wherein the tablet has a hardness of at least 50N, preferably at least 60N, and a disintegration time of less than 60 seconds, preferably less than 40, 30, 20, 15 or 10 seconds.

Typically, the tablet has a hardness of at least 60N, preferably at least 65N, more preferably at least 70N, and even more preferably at least 75N.

In one embodiment of the invention, the orodispersible tablet of the invention comprises a disintegrant, typically in an amount of 0.1 to 20%, 0.5 to 10%, 1 to 10%, 2 to 8% (w/w). Ideally, the disintegrant is a superdisintegrant.

The active agent is typically a hydrophobic active. In this specification, the term “hydrophobic active” should be understood as meaning an active which is poorly soluble or practically insoluble in water and has a solubility of 1 part of solute to 1000 to 10000 parts of water. Generally, the preferred particle size of such actives is in the range of <50 micron, preferably <20, and more preferably <10 microns (to increase the surface area of the drug particles and hence its solubility and dissolution rate). Examples of such actives are the cholesterol-lowering drugs, including the statins simvastatin and atorvastatin, nonsteroidal anti inflammatory agents such as indomethacin, diclofenac, meloxicam and carprofen. Other examples of hydrophobic actives include antihypertensives, anxiolytic agents, anticlotting agents, anticonvulsants, blood glucose lowering agents, decongestants, antihistamines, antitussives, antineoplastics, beta blockers, anti-inflammatory agents, antipsychotic agents, cognitive enhancers, anti-atherosclerotic agents, antiobesity agents, autoimmune disorder agents, anti-impotence agents, and antibacterial and antifungal agents. Generally, the active comprises from 1 to 25%, suitably from 5 to 20%, of the tablet (w/w). Ideally, the active is a high dose active, and is included in the tablet at least 50 mg, 75 mg, 100 mg, 125 mg and 150 mg.

The sugar-based direct compression base may be a sugar, a polyol, or a sugar alcohol. In one preferred embodiment, the DC base is a DC sugar alcohol, ideally DC mannitol or sorbitol. Examples of suitable DC sugar alcohols are Mannitol 100, Mannitol 200, Mannitol 300 and Mannitol 400. Preferably, the DC base is Mannitol 200 or 100. Other types of sugar-based direct compression bases include lactose fast flow, lactose DC, Sorbitol Instant, sucrose, dextrose, xylitol, and maltitol.

Suitably, the sugar-based direct compression base is included in an amount of from 20 to 80%, typically from 30 to 50%, and ideally from 40 to 50% (w/w).

Typically, the MCC base is Avicel. In one preferred embodiment, the MCC base is a silicified MCC base. These bases comprise an intimate physical mixture of colloidal silicon dioxide with microcrystalline cellulose (see for example U.S. Pat. No. 5,585,115). Suitable examples of silicified MCC are ProSolv 50 and ProSolv 90 (Penwest), having an average particle size of 50μ and 90μ, respectively. In a preferred embodiment, the silicified MCC is ProSolv 90.

Suitably, the MCC base is included in an amount of from 20 to 50%, typically from 30 to 50%, and ideally from 40 to 50% (w/w).

In one embodiment, the tablet comprises a disintegrant, preferably a superdisintegrant. Examples of suitable disintegrants and superdisintegrants are provided on pages 12 to 14 of International Patent Application No: PCT/US2003/019527. In a preferred embodiment, the superdisintegrant is selected from the group consisting of: Kollidon-CLSF; ac-di-sol; and Explotab. When included, the disintegrant comprises from 0.1 to 20% of the tablet, ideally from 1 to 10% of the tablet (w/w).

In one embodiment, the tablet contains no disintegrant or superdisintegrant (in this regard, while an MCC base is reported to have disintegrant properties, it is not considered to be a disintegrant).

In one embodiment, the tablet has a friability of less than 1%, as per USP, method, and typically less than 0.6%, and ideally less than 0.2% or 0.1%,

In one preferred embodiment of the invention, the mixture of components additionally comprises a lubricant, typically selected from the group comprising: magnesium stearate; stearic acid, polyethylene glycol, polyoxyethylene-polyoxypropylene block copolymer (poloxamer). Suitably, the lubricant comprises between 0.1% and 5.0%, preferably between 0.2% and 1.0%, of the tablet (w/w).

In another embodiment, the lubricant, instead of or in addition to being included in the tablet formulation, is coated on to the faces of the tabletting punches and dies.

Optionally, the mixture of components includes a flow enhancing agent such as, for example, talc or colloidal silicon dioxide, at from 0.1% to 3.0%, and preferably from 0.1% and 0.5%, of the tablet (w/w). The mixture of components optionally includes a flavouring agent (such as, for example, synthetic oils, natural oils, or extracts from plants or other suitable synthetic or naturally derived flavors), typically at a level ranging from 0.5 to 5% of the tablet (w/w). The mixture of components may also include a surfactant or wetting agent (such as sodium lauryl sulphate, Tweens, Spans), typically at a level of from 0.1 to 3% of the tablet (w/w).

In a particularly preferred embodiment, the tablets of the invention have diameter in the range of 5-20 mm, preferably in the range of 10-15 mm and more preferably 15 mm Typically, the tablet has a diameter of at least 10 mm, at least 11 mm, at least 12 mm, at least 13 mm, and at least 14 mm Preferably, the tablet has a thickness of between 1 and 6 mm, preferably between 1.5-3.5 mm.

In a preferred embodiment of the invention, the compression force employed in the direct compression process is from 6 kN to 20 kN, 8 to 18 kN, or ideally from 8 kN to 15 kN.

In a preferred embodiment of the invention, the tablet is substantially flat-faced. Ideally, the tablet has a bevelled edge. Suitably, the tablet is generally circular, although other shapes of tablets are envisaged such as oval, rectangular, triangular and square.

The tablets of the invention have been found to be particularly suitable for the transmucosal/sublingual delivery of actives, especially poorly permeable actives (for example Class III and IV BCS actives, examples of which would be peptides, proteins, anti cancer agents and other biologic drugs). Without being bound by theory, it has been found that the presence of the sugar-based direct compression excipient in the tablet has the effect of opening tight junctions between the cells in the oral mucosa to aid delivery of poorly permeable drugs. Thus, the administration of a tablet of the invention to the oral cavity, wherein the tablet is maintained in the oral cavity during the disintegration period, facilitates an adequate period of contact between the tablet components and the oral mucosa, thereby having the effect of opening channels in the mucosal cells while also providing bioavailable drug in the vicinity of these cells. Thus, in one embodiment of the invention, the active is a poorly permeable drug, such as a biologic, and wherein the tablet optionally comprises a suitable amount of a permeability enhancer, examples of which will be well known to those skilled in the art.

Thus, in one embodiment, the invention relates to a method for the delivery of a poorly permeable drug via the oral musosa (for example sub-lingual delivery), the method comprising the steps of administering an orodispersible tablet of the invention to a patient in need thereof to an oral cavity of the patient, and keeping the tablet in the oral cavity during the period that the tablet disintegrates, wherein the orodispersible tablet comprises a poorly soluble and/or poorly permeable active and optionally a permeability enhancer typically in an amount of 0.1 to 50% (w/w).

The invention also relates to highly robust orodispersible tablets suitable for use with animals (i.e. non-human mammals) having a hardness of at least 60N and a disintegration time of less than 60, ideally 30 seconds. These tablets are produced using a simplified manufacturing process that employs commercially available excipients and no complicated or expensive manufacturing techniques. The tablets are produced using high amounts of a MCC, ideally a silicified MCC, generally at least 50%, a superdisintegrant (w/w), and an active typically in a non-granulated form. The tablets are directly compressed using high compression forces in the range of 5 to 15 kN to provide tablets having a hardness of at least 60N. Surprisingly, the Applicant has discovered that while the process provides very hard and robust tablets, the tablets also have excellent disintegration times, in most cases less than 20 seconds. The literature indicates that it is extremely difficult, if not impossible, to employ direct compression tabletting to obtain tablets that have acceptable orodispersibility and a hardness of greater than 50N. As an example, WO2004/091585 discloses the use of high amounts of silicified MCC in combination with low substituted hydroxypropyl cellulose (L-HPC) to obtain tablets having a hardness of from 10 to 40N and acceptable orodispersibility characteristics, but indicates that tablets produced having a hardness above 40N did not have acceptable orodispersibility characteristics. Compared with the teaching of WO2004/091585, the Applicant has surprisingly discovered that the use of a superdisintegrant in combination with greater than 50% MCC and compression forces of at least 5, 6, 7, or 8 kN provides a tablet of at least 60N hardness, and in many cases at least 70N hardness, and yet having a dispersion time of less than 20 seconds.

Thus, in another aspect, the invention also relates to a directly compressed orodispersible tablet comprising:
0.1 to 49% of an active agent (w/w);
50 to 99.9% of a microcrystalline cellulose (MCC) direct compression base (w/w); and
1 to 50% of a superdisintegrant or calcium silicate (w/w);
wherein the tablet has a hardness of at least 60N and a disintegration time (DT) of less that 60 seconds, ideally less than 30 or 20 seconds.

In one embodiment, the tablet has a hardness of at least 70N, 80N, 90N, 100N, 110N or 120N, and a DT of less than 50, 40, 30, 20, 15 or 10 seconds.

In one embodiment, the MCC is a silicified MCC having an average particle size of less than 100μ. Ideally, the silicified MCC is a ProSolv, such as ProSolv 50 or ProSolv 90.

Ideally, the tablet is substantially flat-faced, typically with a bevelled edge.

The invention also relates to a method of producing an orodispersible tablet having a hardness of at least 60N and a DT of less than 60 and ideally 30 or 20 seconds, the method comprising the steps of directly compressing a mixture of components at a compression force of at least 5, 6, 7 or 8 kN to form the tablet, wherein the mixture of components comprises 0.1 to 50% of an active agent (w/w), 50 to 99.9% of a microcrystalline cellulose (MCC) direct compression base (w/w), and 1 to 50% of a disintegrant (w/w).

The invention also relates to a method of producing an orodispersible tablet having a hardness of at least 60N and a DT of less than 30 or 20 seconds, the method comprising the steps of directly compressing a mixture of components at a compression force of at least 5, 6, 7, or 8 kN to form the tablet, wherein the mixture of components comprises 0.1 to 50% of an active agent (w/w), 50 to 99.9% of a microcrystalline cellulose (MCC) direct compression base (w/w), and 1 to 20% of a superdisintegrant (w/w).

Typically, the tablets are directly compressed at a compression force of at least 9 kN, 10 kN, 11 kN or 12 kN. Suitably, the tablets are directly compressed using flat-faced toolings.

The methods of the invention involve the tablets being formed in a direct compression process. Suitably, a tablet press is employed. In a preferred embodiment, the direct compression process employs substantially flat faced toolings. Thus, the thickness of the formed tablet will not vary considerably from the centre to the edges (unlike tablets produced using bi-concave toolings which are thicker in the middle that at the edges). Typically, the flat faced toolings have a uniform depth, which will not vary in thickness between the centre and edge by more that +/−5%, preferably 4%, preferably 3%, more preferably 2%, and ideally by more than 1%. Ideally, the tablets have a bevelled edge.

The tablets of the invention generally having a weight of from 50 to 1000 mg, typically from 100 to 700 mg, and ideally from 100 to 500 mg. It will be appreciated that the compression forces required to produce tablets of a defined hardness will vary depending on the size of the tablet. Thus, the methods of the invention may use variable compression forces to achieve a defined tablet hardness depending on the size of the tablet.

The term “direct compression excipient” as used herein will be well known in the art, and refer to excipients, for example MCC or sugar alcohol excipients, which have improved compressibility and/or flowability powders compared to unprocessed excipients in powder forms. The direct compression excipients may be pre-granulated, spray dried, or comprise a polymorphic form that provides improved compressibility and/or flowability.

### DETAILED DESCRIPTION OF THE INVENTION

The examples below provide a number of fast dissolving tablets formed according to the process of the invention. The characteristics of the tablets were determined as follows:

Disintegration time (PharmaTest Disintegration tester PTZ Auto, PTFE Germany)
Hardness or Crushing strength (PharmaTest tablet hardness tester, PTB 411E, Germany)
Uniformity of weight (Sartorius, Model: CP225D)
Thickness (Digital caliper, Workzone UK)

### Friability Tester (PharmaTest, PTFE Germany)

### EXPERIMENTAL

### Carprofen FDDT Formulations Using Prosolv 90

FDDT formulation compositions based on the use of the non sugar, Prosolve 90 silicified microcrystalline cellulose) were developed as a second alternative formulation. Three strengths of carprofen (Cpama S.p.a, Italy, lot number: 101307011) at 20, 50 and 100 mg per unit dose were successfully compressed at high speeds of 49 rpm. FDDTs containing Prosolve in general tend to be thicker than corresponding Mannitol based tablets and have faster disintegration times. However, Prosolve 90 FDDTs tend to be less palatable than sugar based FDDTs and require the addition of sweeteners and higher levels of flavouring to improve palatability. A combination of an MCC (i.e. Prosolve) and a sugar alcohol (dc Mannitol) surprisingly results in improved processability, enhanced hardness and disintegration times, and improved palatability, and hence is preferred particularly for high dose actives at 50 mg and greater.

Other ingredients used in the formulation include colloidal silica, Aerosil 200, the superdisintegrant, Crospovidone (Kollidon® CL-SF) and Magnesium Stearate.

TABLE 1
Examples of Formulation composition for Carprofen
20 mg FDDTs using Prosolv 90 as the filler

| | | B/N | B/N | BN- |
|---|---|---|---|---|
| | Compo- | CAN-032 | CNA-036 | CNA030 |
| | sition | Qty/tab | Qty/tab | Qty/tab |
| Ingredients | % w/w | (mg) | (mg) | mg |
| Carprofen | 13.33 | 20.0 | 50.0 | 100 |
| Prosolv 90 | 77.87 | 116.8 | 292 | 584 |
| Crospovidone | 5.0 | 7.5 | 18.75 | 37.5 |
| Chocolate | 2.0 | 3.0 | 7.5 | 15 |
| Vanilla cream | 0.8 | 1.2 | 3 | 6 |
| flavour | | | | |
| Mg. stearate | 0.5 | 0.75 | 1.875 | 3.75 |
| Aerosil 200 | 0.5 | 0.75 | 1.875 | 3.75 |
| Tab weight | 100 | 150 | 375 | 750 |

FDDTs were compressed at a speed of 49 r.p.m using 10 mm round flat bevelled edge toolings for the 20 and 50 mg Carprofen strengths while 13 mm round flat bevelled edge toolings were used for FDDTs containing 100 mg Carprofen per tablet. Disintegration times were very fast with an average disintegration time in the range of 10.14+/−1.35 to 13+/−2 seconds. FDDTs had an average hardness in the range of 65.5 N±9.8 to 77.77+/−11.84 Newtons. The friability of the tablets was very low at less than the 1% limit. (Table 2)

TABLE 2
Characterisation data for carprofen 20, 50 and 100 mg FDDTs
compressed using prosolv HD90 direct compressible base

| Carprofen FDDT | Weight | Thickness | DT | Hardness | Friability |
|---|---|---|---|---|---|
| batch | (mg) | (mm) | (seconds) | (N) | % |
| CNA032 -20 mg | 148.76 ± | 1.81 ± | 10.14 ± | 70.29 ± | 0.0938 |
| carprofen | 1.16 | 0.01 | 1.34 | 6.60 | |
| CNA036 -50 mg | 380.62 ± | 5.29 ± | 11.0 ± | 65.50 ± | 0.426 |
| carprofen | 3.27 | 0.07 | 2.09 | 9.82 | |
| CNA030 -100 mg | 753.43 ± | 6.47 ± | 13.0 ± | 77.77 ± | 0.2325 |
| carprofen | 9.92 | 0.018 | 2.0 | 11.84 | |

FDDT formulations using Prosolv were also developed for simvastatin and atorvastatin calcium. Two grades of Prosolv were used, Prosolv SMCC 90 and a higher density grade Prosolv SMCC HD 90. FDDT placebos were formulated using both Prosolv SMCC 90 and Prosolv SMCC HD90 (Tables 3 and 4). Simvastatin and atorvastatin FDDTs were formulated using Prosolv SMCC HD 90. A number of disintegrants were studied for the placebo, simvastatin and atorvastatin FDDTs. Tablets were compressed at 10 kN at a target tablet weight of 300 mg using 13 mm round flat faced bevelled edge toolings.

The effect of adding a disintegrant to the Prosolv filler in the placebo FDDTs and the drug containing FDDTs, simvastatin and atorvastatin calcium, shown in Tables 3, 4, 5, and 6 respectively demonstrate a very fast disintegration time of 20 seconds and below for all disintegrants used except for Luquasorb which showed a higher disintegration time of >30 seconds at ˜48 seconds. Surprisingly at the fast disintegration times observed, all tablets formulated were hard with no or negligible friability making them suitable for conventional packaging.

Formulation compositions used for the placebos, simvastatin and atorvastatin FDDTs are listed in corresponding Table 3A, 4A, 5A, 6A respectively.

TABLE 3A
Percent composition of Placebo FDDTs prepared using
Prosolv SMCC 90 as a filler

| | Composition (% w/w) for each batch | | | | | |
|---|---|---|---|---|---|---|
| Ingredients | B110 | B103 | B104 | B105 | B106 | B107 |
| Prosolv | 99.5 | 94.5 | 89.5 | 79.5 | 97.5 | 94.5 |
| SMCC 90 | | | | | | |
| Kollidon | — | 5 | — | — | — | — |
| CLSF | | | | | | |
| Explotab | — | — | 10 | 10 | — | — |
| Calcium | — | — | — | 10 | — | — |
| silicate | | | | | | |
| Luquasorb | — | — | — | — | 2 | — |
| 1161 | | | | | | |
| Ac-di-sol | — | — | — | — | — | 5 |
| Magnesium | 0.5 | 0.5 | 0.5 | 0.5 | 0.5 | 0.5 |
| stearate | | | | | | |
| Total | 100% | 100% | 100% | 100% | 100% | 100% |

TABLE 4A
Percent composition of Placebo FDDTs prepared using
Prosolv SMCC HD90 as a filler

| | Composition (% w/w) for each batch | | | | | |
|---|---|---|---|---|---|---|
| Ingredients | B111 | B112 | B113 | B114 | B115 | B116 |
| Prosolv | 99.5 | 94.5 | 89.5 | 79.5 | 97.5 | 94.5 |
| SMCC HD90 | | | | | | |
| Kollidon | — | 5 | — | — | — | — |
| CLSF | | | | | | |
| Explotab | — | — | 10 | 10 | — | — |
| Calcium | — | — | — | 10 | — | — |
| silicate | | | | | | |
| Luquasorb | — | — | — | — | 2 | — |
| 1161 | | | | | | |
| Ac-di-sol | — | — | — | — | — | 5 |
| Magnesium | 0.5 | 0.5 | 0.5 | 0.5 | 0.5 | 0.5 |
| stearate | | | | | | |
| Total | 100% | 100% | 100% | 100% | 100% | 100% |

TABLE 3
Characteristics of FDDTs prepared using Prosolv SMCC 90, using a
compressional force of 10 kN

| | | Weight | | | | |
|---|---|---|---|---|---|---|
| | | variation | Hardness | Friability | | Thickness |
| Batch No | Disintegrant | (mg) | (N) | (% wt loss) | DT (s) | (mm) |
| B111 | N/A | 292.51 ± 1.65 | 132.46 ± 3.02 | 0.00 | 22 ± 5 | 1.97 ± 0.02 |
| B112 | Kollidon | 295.97 ± 2.68 | 96.98 ± 2.91 | 0.00 | 11 ± 5.57 | 1.99 ± 0.01 |
| | CLSF (5%) | | | | | |
| B113 | Explotab | 299.18 ± 0.59 | 141.11 ± 20.42 | 0.00 | 7.33 ± 0.58 | 2.06 ± 0.01 |
| | (10%) | | | | | |
| B114 | Explotab + | 302.24 ± 0.20 | 76.91 ± 1.96 | 0.00 | 5.33 ± 1.15 | 2.01 ± 0.01 |
| | Ca Silicate | | | | | |
| | (5% + 5%) | | | | | |
| B115 | Luquasorb | 301.20 ± 1.95 | 119.55 ± 6.98 | 0.1 | 47.67 ± 3.06 | 2.04 ± 0.02 |
| | 1161 (2%) | | | | | |
| B116 | Ac-di-sol | 298.16 ± 2.24 | 128.20 ± 8.89 | 0.00 | 7 ± 0 | 2.05 ± 0.02 |
| | (5%) | | | | | |

TABLE 4
Characteristics of FDDTs formulated using Prosolv SMCC HD 90 at a
compressional force of 10 kN

| | | Weight | | Friability | | Tablet |
|---|---|---|---|---|---|---|
| Batch | | variation | Hardness | (% wt | | Thickness |
| No | Disintegrant | (mg) | (N) | loss) | DT (s) | (mm) |
| B110 | None | 290.92 ± 0.94 | 161.62 ± 13.04 | 0.00 | 163.33 ± 47.44 | 1.99 ± 0.00 |
| B103 | Kollidon | 296.31 ± 5.08 | 168.48 ± 5.55 | 0.00 | 20 ± 3.46 | 2.04 ± 0.04 |
| | CLSF (5%) | | | | | |
| B104 | Explotab | 296.07 ± 4.20 | 139.10 ± 8.66 | 0.00 | 6.67 ± 0.58 | 1.97 ± 0.06 |
| | (10%) | | | | | |
| B105 | Explotab | 299.5 ± 1 | 100.01 ± 3.58 | 0.33 | 9.67 ± 0.58 | 2.00 ± 0.01 |
| | (10%) + Ca | | | | | |
| | Silicate | | | | | |
| | (10%) | | | | | |
| B106 | Luquasorb | 298.47 ± 0.55 | 158.76 ± 9.17 | 0.00 | 48.67 ± 1.53 | 2.00 ± 0.01 |
| | 1161 (2%)* | | | | | |
| B107 | Ac-di-sol | 299.67 ± 3.8 | 171.81 ± 15.81 | 0.00 | 14 ± 1 | 2.03 ± 0.01 |
| | (5%) | | | | | |

TABLE 5A
Percent composition of simvastatin FDDTs
prepared using Prosolv SMCC HD90 as a filler

| | Composition (% w/w) for each batch | | | |
|---|---|---|---|---|
| Ingredients | B117 | B118 | B127 | B120 |
| Simvastatin | 6.67 | 6.67 | 6.67 | 6.67 |
| Prosolv SMCC | 86.23 | 81.23 | 81.23 | 86.23 |
| HD90 | | | | |
| Kollidon CLSF | 5 | — | — | — |
| Explotab | — | 10 | — | — |
| Calcium silicate | — | — | 10 | — |
| Ac-di-sol | — | — | — | 5 |
| Magnesium | 0.5 | 0.5 | 0.5 | 0.5 |
| stearate | | | | |
| Vanilla | 0.8 | 0.8 | 0.8 | 0.8 |
| Raspberry | 0.8 | 0.8 | 0.8 | 0.8 |
| Total | 100% | 100% | 100% | 100% |

TABLE 5
Characteristics of Simvastatin FDDTs formulated using Prosolv SMCC HD
90 at a compressional force of 10 kN

| | | Weight | | Friability | | |
|---|---|---|---|---|---|---|
| Batch | | variation | Hardness | (% wt | | Thickness |
| No | Disintegrant | (mg) | (N) | loss) | DT (s) | (mm) |
| B117 | Kollidon | 297.7 ± 0.71 | 109.95 ± 11.99 | 0.1 | 9.33 ± 0.58 | 2.08 ± 0.01 |
| | CLSF (5%) | | | | | |
| B118 | Explotab | 295.87 ± 2.68 | 72.28 ± 3.57 | 0.2 | 8 ± 2 | 2.00 ± 0.01 |
| | (10%) | | | | | |
| B120 | Ac-di-sol | 301.59 ± 0.37 | 94.78 ± 1.14 | 0.3 | 10 ± 1 | 2.12 ± 0.04 |
| | (5%) | | | | | |
| B127 | Calcium | 299.33 ± 1.53 | 76.53 ± 2.51 | 0.03 | 8.67 ± 0.58 | 2.00 ± 0.03 |
| | silicate | | | | | |
| | (10%) | | | | | |

TABLE 6A
Percent composition of Atorvastatin tablets
prepared using Prosolv SMCC HD90 as a filler

| | Composition (% w/w) for each batch | | | |
|---|---|---|---|---|
| Ingredients | B122 | B123 | B128 | B125 |
| Atorvastatin | 6.67 | 6.67 | 6.67 | 6.67 |
| Prosolv SMCC | 86.23 | 81.23 | 81.23 | 86.23 |
| HD90 | | | | |
| Kollidon CLSF | 5 | — | — | — |
| Explotab | — | 10 | — | — |
| Calcium silicate | — | — | 10 | — |
| Ac-di-sol | — | — | — | 5 |
| Magnesium | 0.5 | 0.5 | 0.5 | 0.5 |
| stearate | | | | |
| Vanilla | 0.8 | 0.8 | 0.8 | 0.8 |
| Raspberry | 0.8 | 0.8 | 0.8 | 0.8 |
| Total | 100% | 100% | 100% | 100% |

TABLE 6
Characteristics of Atorvastatin calcium FDDTs formulated using Prosolv
SMCC HD 90 at a compressional force of 10 kN

| | | Weight | | Friability | | |
|---|---|---|---|---|---|---|
| Batch | | variation | Hardness | (% wt | | Thickness |
| No | Disintegrant | (mg) | (N) | loss) | DT (s) | (mm) |
| B122 | Kollidon | 297.42 ± 1.66 | 103.20 ± 4.62 | 0.00 | 13.33 ± 2.08 | 2.08 ± 0.02 |
| | CLSF (5%) | | | | | |
| B123 | Explotab | 297.58 ± 0.80 | 73.11 ± 1.24 | 0.00 | 5.67 ± 1.15 | 2.06 ± 0.02 |
| | (10%) | | | | | |
| B125 | Ac-di-sol | 300.48 ± 1.66 | 95.62 ± 1.97 | 0.33 | 8.67 ± 1.53 | 2.05 ± 0.0 |
| | (5%) | | | | | |
| B128 | Ca Silicate | 294.50 ± 0.25 | 73.95 ± 6.04 | 0.03 | 9 ± 2 | 2.01 ± 0.01 |
| | (10%) | | | | | |

### Formulation Development of FDDT Formulations Using a Mixture of Prosolv SMCC 90 and a Sugar DC Base.

FDDT formulations containing simvastatin, or carprofen were formulated using a mixture of 2 direct compressible bases, a cellulose based DC, Prosolv or Avicel and a sugar based DC, Mannitol. Prosolv SMCC 90 and Avicel PH101 were used and the Mannitol used was Mannitol 200.

Simvastatin FDDTs were compressed at 8-10 kN, at a target tablet weight of 300 mg per 20 mg dose of simvastatin, using 13 mm round flat faced bevelled edge toolings. The disintegrant used was Kollidon CLSF at 5% w/w. Formulation composition and characteristics of FDDTs formulated are given in Table 7 and 8 respectively.

TABLE 7
Composition of simvastatin FDDT formulations
containing 20 mg of simvastatin per 300 mg tablet.

| | Composition | B/N 2008/037 |
|---|---|---|
| Ingredients | % w/w | Qty/tab (mg) |
| simvastatin | 6.7 | 20.0 |
| Prosolv 90 | 43.1 | 129.3 |
| Mannitol 100 | 43.1 | 129.3 |
| Crospovidone, | 5.0 | 15 |
| K-CLSF | | |
| Raspberry | 0.8 | 2.4 |
| Mint | 0.8 | 2.4 |
| Mg. stearate | 0.5 | 1.5 |
| Tab weight | 100 | 300 |

The FDDT weights were in the range of average weight±5% (Table 8). A very fast disintegration time of 16.17 seconds was observed for the FDDTs, related to the combination of the water soluble mannitol, the fast dispersing Prosolv and superdisintegrant crospovidone and flat bevelled edge toolings used. The FDDTs produced were robust with an average tablet hardness was 64.5N and the FDDT had a friability of 0, making them suitable for conventional packaging.

TABLE 8
Characterisation data for 20 mg simvastatin
FDDTs, BN 2008/037, compressed at 49 r.p.m

| | Weight | Thickness | DT | Hardness | Friability |
|---|---|---|---|---|---|
| | (mg) | (mm) | (secs) | (N) | % |
| Batch no. | 300.85 ± 2.9 | 2.2 ± 0.01 | 16.17 ± 1.6 | 64.5 ± 5.1 | 0.0 |
| 2008/037 | | | | | |

### Formulation Development of 20 and 100 mg Carprofen FDDTs Using a Combination of Mannitol 200 and Avicel 101 Blends

Two ratios of Mannitol 200 to microcrystalline cellulose, Avicel PH 101, were used to formulate FDDTs containing carprofen at 20 mg per unit. FDDTs were produced at a compression speed of 7 and at the higher compression speed of 49 r.p.m. using a compression force of 10 KN and 10 mm round flat bevelled edged toolings. No disintegrant was included in these batches. Compositions used and FDDT characteristics are given in Tables 9 and 10.

TABLE 9
Formulation composition for Carprofen 20 mg FDDT
using Mannitol 200 and Avicel PH101 at 2 ratios

| | Batch 2008/041 | | Batch 2008/049 | |
|---|---|---|---|---|
| Ingredients | % w/w | Qty/tab mg | % w/w | Qty/tab mg |
| Carprofen | 13.33 | 20 | 13.33 | 20 |
| Mannitol 200 | 46.0 | 69 | 63.58 | 95.37 |
| Avicel PH101 | 38.77 | 58.2 | 21.192 | 32.88 |
| Aerosil 200 | 0.86 | 1.29 | 0.86 | 1.29 |
| Mg Stearate | 1.04 | 1.56 | 1.04 | 1.56 |
| Tab weight | 100 | 150 | 100 | 150 |

The FDDT weights were in the range of average weights±5%. Disintegration times for both batches were 39.3 seconds for 2008/041 and 33.8 seconds for 2008/049 which are well below 60 seconds. Avicel has a lower solubility than Mannitol and in the absence of a disintegrant, it is surprising that B/N 2008/041 and B/N 2008/049 tablets had a fast disintegration time of below 60 seconds. The average tablet hardness of these tablets were 67 N and 62.6 N and both batches of tablets passed friability tests with low friability values <0.2%, well below 1% USP limit for conventional tablets.

TABLE 10
Characterisation data for 20 and 100 mg Carprofen FDDT using
Mannitol 200 & Avicel PH101 and compressed at 49 r.p.m

| | Weight | Thickness | DT | Hardness | Friability |
|---|---|---|---|---|---|
| Batch no. | (mg) | (mm) | (secs) | (N) | % |
| 2008/041 | 148.11 ± | 1.685 ± | 39.33 ± | 67.15 ± | 0.142 |
| | 1.16 | 0.007 | 7.50 | 9.15 | |
| 2008/049 | 146.60 ± | 1.713 ± | 33.83 ± | 62.60 ± | 0.161 |
| | 0.53 | 0.008 | 5.23 | 11.54 | |
| 2008/094 | 742.10 ± | 4.040 ± | 25.17 ± | 64.77 ± | 0.5743 |
| | 3.78 | 0.016 | 2.40 | 2.23 | |

The formulation composition used for Batch 2008/049 was used to compress FDDTs containing Carprofen at 100 mg per tablet, batch number 2008/094. The formulation, at a target tablet weight of 750 mg, was compressed on a Piccola eight station tablets press using a compressional force of 9 KN using a speed of 7 r.p.m and 49 r.p.m. Larger toolings of 15 mm round flat bevelled edged were used for the 100 mg FDDTs. The characteristics of these 100 mg Carprofen FDDTs, BN 2008/094, are given in Table 10.

All FDDTs were within the range of average weight±5.09%. Disintegration times were lower than 30 seconds with an average disintegration time of 25.2 seconds, probably a result of the larger diameter toolings. The tablets had an average hardness of 64.8 N±2.23, similar to the hardness observed for the 20 mg FDDTs using the same formulation. FDDTs passed friability tests with a friability of 0.57% below the 1% USP limit.

The invention is not limited to the embodiment hereinbefore described which may be varied in construction and detail without departing from the spirit of the invention.

1-58. (canceled)

59. A method of producing an orodispersible tablet comprising the steps of directly compressing a mixture of components at a compression force of at least 51 kN to form the tablet, wherein the mixture of components comprises 0.1 to 50% of an ungranulated active agent (w/w), 10 to 80% of a sugar-based direct compression base (w/w); and 10 to 80% of a microcrystalline cellulose (MCC) direct compression base (w/w).

60. A method as claimed in claim 59 in which the mixture of components comprises 0.1 to 30% of an ungranulated active agent (w/w), 30 to 50%, of a sugar-based direct compression base; and 30 to 50%, of a microcrystalline cellulose (MCC) direct compression base.

61. A method as claimed in claim 60 in which the mixture of components comprises 0.1 to 30% of an ungranulated active agent (w/w), 40 to 50%, of a sugar-based direct compression base; and 40 to 50%, of a microcrystalline cellulose (MCC) direct compression base.

62. A method as claimed in claim 59 in which the sugar-based direct compression base is a direct compression sugar alcohol.

63. A method as claimed in claim 59 in which the tablets are directly compressed at a compression force of at least 10 kN.

64. A method as claimed in claim 59 in which the MCC direct compression base is selected from silicified MCC and Avicel.

65. A method as claimed in claim 59 which is a method for producing orodispersible tablets having a hardness of at least 50N and a disintegration time of less than 30 seconds.

66. A method as claimed in claim 59 which is a method for producing orodispersible tablets having a friability of less than 1%.

67. A method of producing an orodispersible tablet according to claim 59, the tablet having a hardness of at least 50N and a disintegration time of less than 30 seconds, the method comprising the steps of directly compressing a mixture of components at a compression force of at least 5 kN to form the tablet, wherein the mixture of components comprises 0.1 to 50% of an ungranulated active agent (w/w), 10 to 80% of a direct compression mannitol base (w/w); and 10 to 80% of a microcrystalline cellulose (MCC) direct compression base (w/w).

68. A method of producing an orodispersible tablet according to claim 59, the tablet having a hardness of at least 50N and a disintegration time of less than 30 seconds, the method comprising the steps of directly compressing a mixture of components at a compression force of at least 5 kN to form the tablet, wherein the mixture of components comprises 0.1 to 50% of an ungranulated active agent (w/w), 30 to 50% of a direct compression mannitol base (w/w); and 30 to 50% of a microcrystalline cellulose (MCC) direct compression base (w/w).

69. A method as claimed in claim 59 in which the sugar-based direct compression excipient is a non-filamentous excipient.

70. A directly compressed orodispersible tablet comprising:
0.1 to 50% of a non-granulated active agent (w/w);
10 to 80% of a sugar-based direct compression base (w/w); and
10 to 80% of a microcrystalline cellulose (MCC) direct compression base (w/w).

71. A tablet as claimed in claim 70 and having a hardness of at least 50N and a disintegration time of less than 30 seconds.

72. A tablet as claimed in claim 70 and comprising:
0.1 to 20% of a non-granulated active agent (w/w);
30 to 50%, of a DC sugar alcohol (w/w);
30 to 50% of a MCC (w/w); and
optionally, one or more of a lubricant, a disintegrant, flavouring agent, and a flow enhancer,
wherein the tablet has a hardness of at least 50N and a disintegration time of less than 60 seconds.

73. A tablet as claimed in claim 72 and having a hardness of at least 60N and a disintegration time of less than 30 seconds.

74. A tablet as claimed in claim 70 and including a superdisintegrant in an amount of from 1 to 20%.

75. A tablet as claimed in claim 70 in which the active agent is a hydrophobic active.

76. A tablet as claimed in claim 70 in which the sugar-based direct compression base is a DC sugar alcohol.

77. A tablet as claimed in claim 70 in which the MCC base is Avicel or silicified MCC.

78. A tablet as claimed in claim 70 in which the active is a poorly permeable active, and further includes a permeability enhancer in an amount of 0.1% to 50% (w/w)

79. A tablet as claimed in claim 78 in which the permeability enhancer is included in the tablet in an amount of 0.1-10% (w/w).

80. A method for the delivery of a poorly soluble drug via the oral musosa, the method comprising the steps of administering an orodispersible tablet of claim 70 to a patient in need thereof to an oral cavity of the patient, and keeping the tablet in the oral cavity during the period that the tablet disintegrates, wherein the orodispersible tablet comprises a poorly permeable active and optionally a permeability enhancer in an amount of from 0.1% to 50% (w/w).

81. A directly compressed orodispersible tablet comprising:
0.1 to 49% of a non-granulated active agent (w/w);
50 to 99.9% of a microcrystalline cellulose (MCC) direct compression base (w/w); and
1 to 50% of a superdisintegrant or calcium silicate (w/w);
wherein the tablet has a hardness of at least 60N and a disintegration time (DT) of less than 30 seconds.

82. A tablet as claimed in claim 81 having a hardness of at least 70N.

83. A tablet as claimed in claim 81 having a disintegration time of less than 20 seconds.

84. A tablet as claimed in claim 81 in which the superdisintegrant is selected from a povidone (i.e. Kollidon-CLSF), ac-di-sol, and Explotab or the like.
